(19) United States
(12) Patent Application Publication
(10) Pub. No.: US 2022/0340836 A1
(43) Pub. Date: Oct. 27, 2022

(54) METHOD OF PREPARATION WHITE EMULSIFIED SCENTED CROCODILE OIL

(71) Applicant: Liang Chieh WEE, Singapore (SG)

(72) Inventor: Liang Chieh WEE, Singapore (SG)

(21) Appl. No.: 17/765,900

(22) PCT Filed: Jul. 8, 2020
(86) PCT No.: PCT/SG2020/050391

§ 371 Date: Apr. 1, 2022

(30) Foreign Application Priority Data
Oct. 2, 2019 (SG) 10201909221P

(51) Int. Cl. C11B 1/14; C11B 3/00

(52) U.S. Cl. CPC C11B 1/14; C11B 3/008

(57) ABSTRACT

The present invention relates to a method of preparing a crocodile oil to reduce unpleasant odour comprising: preparing crocodile fats and mucosal tissues; adding water to the crocodile fats in the ratio of water to crocodile fats at 1:15 by weight, heating the crocodile fats until boiling; adding the mucosal tissues of crocodiles into the boiled crocodile fats until an oil is rendered from the crocodile fats; cooling down the crocodile fats to room temperature to obtain a crude crocodile oil; adding water, plant essence and emulsifier to the crude crocodile oil and mixing to obtain a crude crocodile oil mixture; filtering out oil dregs from the crude crocodile oil mixture; passing the remaining white liquid through a high pressure homogenizer to obtain a scented crocodile oil; and adding 15 to 20 ml of ginger oil to every 5000 ml of the scented crocodile oil and obtain the crocodile oil.

### FIELD OF INVENTION

The invention relates a process of preparation of a crocodile oil, specifically a mixture process comprising or consisting of two or more different oil for reducing an odour, in particular an unpleasant odour, or as an auxiliary for reducing an unpleasant odour.

### BACKGROUND OF THE INVENTION

Unpleasant odours arise in many industrially produced basic substances used in cosmetic, body care, household or cleaning products, in particular in basic substances used in detergents, for example for laundry detergents and fabric softeners, or in body care products, for example for soaps and cosmetics. The use of specific composition in mixture preparations produce unpleasant odours and thus cause upset to consumers, hence consumers tends to avoid buying the product having such odours event its bring benefited to their health or wellbeing.

The process of reducing odours, in particular unpleasant odours is difficult to treat and solve in terms of cosmetic products. In particular, the specific character of a respective unpleasant odour of crocodile's oil restricts the use of diverse preparations, in cosmetic products. Hence, there is a need a substances or materials which are able to reduce the intensity of unpleasant odours of crocodile's oil or even to completely eliminate the unpleasant odour.

Ginger is widely used as a spice or a medicine, and ginger oil is also sometimes used for reducing chest pain, low back pain, stomach pain, and pain in some people with a form of arthritis. However, it's not commonly used in cosmetic product or to reduce the unpleasant odours. The proposed invention able to cover or eliminate the unpleasant odour by using the ginger oil such that it can easily accepted by consumers.

### SUMMARY OF THE INVENTION

One embodiment of a present invention relates a method of preparation of a crocodile oil, comprising steps of: allocating crocodile fats and mucosal tissues; washing the crocodile fats; placing the washed crocodile fats into a boiler means; adding water in parts by weight of crocodile fats to water, in the ratio of 1:15; placing the boiler means on high heat until the crocodile fats is boiled; adding the mucosal tissues of crocodiles into the boiled crocodile fats; placing the boiler means on low heat temperature, and cook it in water using a cooking means, until an oil is rendered from the crocodile fats; cooling down the boiler to a room temperature to obtained a crude crocodile oil from the oil; mixing the crude crocodile oil using a mixing means, with a water, plant essence and emulsifier in parts by weight, in the ratio of 1:1:0.5:0.5; filtering out oil dregs from the mixture, and keeping remaining white liquid; and passing the white liquid obtained through a high pressure homogenizer to obtain white an emulsified scented crocodile oil, characterised in that at least 15 to 20 ml of ginger oil added with against every 5000 ml of the scented crocodile oil, such that the Ginger oil able to reduce odours of the scented crocodile oil or adds fragrance to the mixture.

Preferably, the steps of allocating is in parts by weight, 70-85 parts of crocodile fats and 5-10 parts of mucosal tissues.

Preferably, the steps of placing the boiler means on low heat temperature, and cook it in water for 15-18 hours using the cooking means.

Preferably, the steps of mixing the crude crocodile oil using the mixing means is for 6 hours.

Preferably, the crocodile fats is obtained from flanks or belly of a crocodile.

Preferably, the low heat is between 98° C.-360° C.

Preferably, the high pressure homogenizer provides pressure treatment of 30-110 MPa.

The present invention consists of features and a combination of parts hereinafter fully described and illustrated in the accompanying drawings, it being understood that various changes in the details may be made without departing from the scope of the invention or sacrificing any of the advantages of the present invention.

### DETAILED EMBODIMENTS OF INVENTION

To further clarify various aspects of some embodiments of the present invention, a more particular description of the invention will be rendered by references to specific embodiments thereof. It is appreciated that these example depict only typical embodiments of the invention and are therefore not to be considered limiting of its scope.

The invention relates a process of preparation of a crocodile oil, specifically a mixture process comprising or consisting of two or more different oil for reducing an odour, in particular an unpleasant odour, or as an auxiliary for reducing an unpleasant odour by using Ginger or Ginger oil to cover or eliminate the unpleasant odour such that it can easily accepted by consumers

Ginger or Ginger oil substances or materials are of great advantage which are able to reduce the intensity of unpleasant odours or even to completely eliminate the unpleasant odour, without themselves being of a considerable a factory-perfume intensity. The Ginger or Ginger oil substances may advantageously neutralise unpleasant odours without masking the respective unpleasant odour merely by their own specific odour. However, the Ginger or Ginger oil substances could be increased such that the mixture has the Ginger or Ginger oil smell depending on preferences.

The use of Ginger or Ginger oil substances has or could have the advantage that to fragrance or perfume objects, it is possible to use preparations or products having unpleasant odours, for example perfume oils of any fragrance direction.

A reduction of unpleasant odours is achieved by using a specifically developed Ginger oil which itself has its own quite specific fragrance type. In this case, the unpleasant odour impressions are masked or “covered” by means of substances of crocodile oil odour.

Preferably, one embodiment includes using method or process of having at least 0.3-0.5% of Ginger oil of the total content Crocodile Oil in a container, such that the Ginger oil able to reduce odours of the Crocodile Oil or adds fragrance to the mixture.

Another embodiment includes using method or process of adding at least 15 to 20 ml of Ginger oil with against every 5000 ml of Crocodile Oil, such that the Ginger oil able to reduce odours of the Crocodile Oil or adds fragrance to the mixture. The invention provides for the method of preparation of a kind of white emulsified scented crocodile oil, to address the shortcomings present in available crocodile oils in the market, such as complicated preparation processes, overpowering fishy smell and restricted quality.

The invention is discussed in detail below by way of embodiments.

### Embodiment 1

(1) Prepare, in parts by weight, 70 parts of fats from the belly of crocodiles and 5 parts of the mucosal tissues of crocodiles. Wash crocodile belly fats, crocodile blood vessels and the mucosal tissues thoroughly and set aside.

(2) Put the crocodile fats into a boiler. Add in 1050 parts of water. Place on high heat until boiled.

(3) Add the mucosal tissues of crocodiles mentioned in step (1) into the crocodile fats mentioned in step (2). Lower the heat and maintain the temperature at between 98° C. and 360° C. Cook it in water for 16 hours, until all the oil comes out from the crocodile fats.

(4) Cool down to room temperature. Crude crocodile oil is obtained.

5) In a mixer, combine the crocodile oil obtained in step (4) with water, plant essence and emulsifier in parts by weight, in the ratio of 1:1:0.5:0.5. Mix for 6 hours. Filter out oil dregs, and keep the remaining milky liquid.

(6) Pass the white liquid obtained from step (5) through a high pressure homogenizer at a pressure treatment of 100 MPa to obtain white emulsified scented crocodile oil.

(7) Adding at least 15 to 20 ml of Ginger oil with against every 5000 ml of Crocodile Oil, such that the Ginger oil able to reduce odours of the Crocodile Oil or adds fragrance to the mixture.

### Embodiment 2

(1) Prepare, in parts by weight, 75 parts of fats from the belly of crocodiles and 9 parts of the mucosal tissues of crocodiles. Wash crocodile fats and the mucosal tissues thoroughly and set aside.

(2) Put the crocodile fats into a boiler. Add in 1125 parts of water. Place on high heat until boiled.

(3) Add the mucosal tissues of crocodiles mentioned in step (1) into the crocodile fats mentioned in step (2). Lower the heat and maintain the temperature at between 98° C. and 360° C. Cook it in water for 16.5 hours, until all the oil comes out from the crocodile fats.

(4) Cool down to room temperature. Crude crocodile oil is obtained.

(5) In a mixer, combine the crocodile oil obtained in step (4) with water, plant essence and emulsifier in parts by weight, in the ratio of 1:1:0.5:0.5. Mix for 6 hours. Filter out oil dregs, and keep the remaining milky liquid.

(6) Pass the white liquid obtained from step (5) through a high pressure homogenizer at a pressure treatment of 105 MPa to obtain white emulsified scented crocodile oil.

(7) Adding at least 15 to 20 ml of Ginger oil with against every 5000 ml of Crocodile Oil, such that the Ginger oil able to reduce odours of the Crocodile Oil or adds fragrance to the mixture.

### Embodiment 3

(1) Prepare, in parts by weight, 80 parts of fats from the belly of crocodiles and 7 parts of the mucosal tissues of crocodiles. Wash crocodile fats and the mucosal tissues thoroughly and set aside.

(2) Put the crocodile fats into a boiler. Add in 1200 parts of water. Place on high heat until boiled.

(3) Add the mucosal tissues of crocodiles mentioned in step (1) into the crocodile fats mentioned in step (2). Lower the heat and maintain the temperature at between 98° C. and 360° C. Cook it in water for 17 hours, until all the oil comes out from the crocodile fats,

(4) Cool down to room temperature. Crude crocodile oil is obtained.

(5) In a mixer, combine the crocodile oil obtained in step (4) with water, plant essence and emulsifier in parts by weight, in the ratio of 1:1:0.5:0.5. Mix for 6 hours. Filter—out oil dregs, and keep the remaining milky liquid.

(6) Pass the white liquid obtained from step (5) through a high pressure homogenizer at a pressure treatment of 108 MPa to obtain white emulsified scented crocodile oil.

(7) Adding at least 15 to 20 ml of Ginger oil with against every 5000 ml of Crocodile Oil, such that the Ginger oil able to reduce odours of the Crocodile Oil or adds fragrance to the mixture.

### Embodiment 4

(1) Prepare, in parts by weight, 85 parts of fats from the belly of crocodiles and 5 parts of the mucosal tissues of crocodiles. Wash crocodile and the mucosal tissues thoroughly and set aside.

(2) Put the crocodile fats into a boiler. Add in 1275 parts of water. Place on high heat until boiled.

(3) Add the crocodile blood, crocodile blood vessels and mucosal tissues of crocodiles mentioned in step (1) into the crocodile fats mentioned in step (2). Lower the heat and maintain the temperature at between 98° C. and 360° C. Cook it in water for 18 hours, until all the oil comes out from the crocodile fats;

(4) Cool down to room temperature. Crude crocodile oil is obtained.

(5) In a mixer, combine the crocodile oil obtained in step (4) with water, plant essence and emulsifier in parts by weight, in the ratio of 1:1:0.5:0.5. Mix for 6 hours. Filter out oil dregs, and keep the remaining milky liquid.

(6) Pass the white liquid obtained from step (5) through a high pressure homogenizer at a pressure treatment of 110 MPa to obtain white emulsified scented crocodile oil.

(7) Adding at least 15 to 20 ml of Ginger oil with against every 5000 ml of Crocodile Oil, such that the Ginger oil able to reduce odours of the Crocodile Oil or adds fragrance to the mixture.

The aforesaid crocodile fats refer to fats obtained from the flanks or belly of crocodiles.

Temperature of the low heat referred to in step (3) above is maintained between 98° C. and 360° C.

Pressure used in the high pressure homogenizer referred to in step (6) above is between 100 MPa and 110 MPa.

The aforesaid crocodile oil is notably good for skin beautification, treatment of skin burns, scalds and frostbites, promotion of scar healing, delaying of skin ageing, and erasing of wrinkles.

From the foregoing description of the structure of the invention, the invention possesses the following advantages over existing technology:

1. Crocodile fat is the main ingredient of the invention. Mucosal tissue of crocodiles, which contains protease good for the human body in terms of skin whitening, healing and erasing of wrinkles, is added. Maximum extraction of protease can only be done through heating in water over low heat. When combined with the unsaturated fatty acids extracted from crocodile fats, including 9, 12-octadecenoic acid, 9-octadecenoic acid and 9-hexadecenoic acid etc., it is notably good for skin beautification, treatment of skin burns, scalds and frostbites, promotion of scar healing, delaying of skin ageing, and erasing of wrinkles.

2. The crude crocodile oil in the invention, when mixed with water and plant essence, will react with the plant essence to produce white emulsified coconut oil that effectively removes the fishy smell. When passed through a high pressure homogenizer, liquid scented crocodile oil is obtained.

The present invention may be embodied in other specific forms without departing from its essential characteristics. The described embodiments are to be considered in all respects only as illustrative and not restrictive. The scope of the invention is, therefore indicated by the appended claims rather than by the foregoing description. All changes, which come within the meaning and range of equivalency of the claims, are to be embraced within their scope.

Throughout this specification, unless the context requires otherwise, the word “comprise”, or variations such as “comprises” or “comprising”, will be understood to imply the inclusion of a stated step or element or integer or group of steps or elements or elements or integers, but not the exclusion of any other step or element or integer or group of steps, elements or integers. Thus, in the context of this specification, the term “comprising” is used in an inclusive sense and thus should be understood as meaning “including principally, but not necessarily solely”.

The foregoing are merely detailed descriptions of the modes of carrying out the invention. The design concept of the invention is not limited’ to the above, and any immaterial variations made to the invention using the concept shall constitute an infringement of the protection scope of the invention.

1. A method of preparation of a crocodile oil, comprising steps of:
allocating crocodile fats and mucosal tissues;
washing the crocodile fats;
placing the washed crocodile fats into a boiler means;
adding water in parts by weight of crocodile fats to water, in the ratio of 1:15;
placing the boiler means on high heat until the crocodile fats is boiled;
adding the mucosal tissues of crocodiles into the boiled crocodile fats;
placing the boiler means on low heat temperature, and cook it in water using a cooking means, until an oil is rendered from the crocodile fats;
cooling down the boiler to a room temperature to obtained a crude crocodile oil from the oil;
mixing the crude crocodile oil using a mixing means, with a water, plant essence and emulsifier in parts by weight, in the ratio of 1:1:0.5:0.5;
filtering out oil dregs from the mixture, and keeping remaining white liquid; and
passing the white liquid obtained through a high pressure homogenizer to obtain white an emulsified scented crocodile oil,
characterised in that at least 15 to 20 ml of ginger oil added with against every 5000 ml of the scented crocodile oil, such that the Ginger oil able to reduce odours of the scented crocodile oil or adds fragrance to the mixture.

2. The method of preparation of a crocodile oil according to claim 1, wherein the steps of allocating is in parts by weight, 70-85 parts of crocodile fats and 5-10 parts of mucosal tissues.

3. The method of preparation of a crocodile oil according to claim 1, wherein the steps of placing the boiler means on low heat temperature, and cook it in water for 15-18 hours using the cooking means.

4. The method of preparation of a crocodile oil according to claim 1, wherein the steps of mixing the crude crocodile oil using the mixing means is for 6 hours.

5. The method of preparation of a crocodile oil according to claim 1, wherein the crocodile fats is obtained from flanks or belly of a crocodile.

6. The method of preparation of a crocodile oil according to claim 1, wherein the low heat is between 98° C.-360° C.

7. The method of preparation of a crocodile oil according to claim 1, wherein the high pressure homogenizer provides pressure treatment of 30-110 MPa.

8. The method of preparation of a crocodile oil according to claim 1, wherein the Crocodile oil is fragrance.

9. The method of preparation of a crocodile oil according to claim 1, wherein the Crocodile oil is in White or Transparent.
